(19) United States
(12) Patent Application Publication
(10) Pub. No.: US 2023/0072879 A1
(43) Pub. Date: Mar. 9, 2023

(54) SYSTEMS AND METHODS FOR HYBRID IMAGING AND NAVIGATION

(71) Applicant: Noah Medical Corporation, San Carlos, CA (US)

(72) Inventors: Piotr Robert Slawinski, South San Francisco, CA (US); Kyle Ross Danna, Scotts Valley, CA (US); Jian Zhang, Redwood City, CA (US)

(21) Appl. No.: 18/054,824

(22) Filed: Nov. 11, 2022

Related U.S. Application Data
(63) Continuation of application No. PCT/US2021/035502, filed on Jun. 2, 2021.
(60) Provisional application No. 63/034,142, filed on Jun. 3, 2020.

(51) Int. Cl. A61B 34/20

(52) U.S. Cl. CPC A61B 34/20; A61B 2034/105; A61B 2034/2051

(57) ABSTRACT

A method is provided for navigating an endoscopic device through an anatomical luminal network of a patient. The method comprises: (a) commanding a distal tip of an articulating elongate member to move along a pre-determined path; (b) concurrent with (a), collecting positional sensor data and kinematics data; and (c) computing an estimated roll angle based on the positional sensor data and the kinematics data.

### REFERENCE

This application is a Continuation Application of International Application No. PCT/US2021/035502, filed Jun. 2, 2021, which claims the benefit of U.S. Provisional Application No. 63/034,142, filed Jun. 3, 2020, which applications are incorporated herein by reference.

### BACKGROUND OF THE INVENTION

Early diagnosis of lung cancer is critical. The five-year survival rate of lung cancer is around 18% which is significantly lower than next three most prevalent cancers: breast (90%), colorectal (65%), and prostate (99%). A total of 142,000 deaths were recorded in 2018 due to lung cancer.

Robotics technology has advantages that can be incorporated into endoscopes for a variety of applications, including bronchoscope. For example, by exploiting soft deformable structures that are capable of moving effectively through a complex environment like inside the main bronchi, one can significantly reduce pain and patient discomfort. However, the guidance of such robotic endoscopes may still be challenging due to the insufficient accuracy and precision of sensing and detecting the complexity and dynamic environment inside the patient body.

### SUMMARY OF THE INVENTION

A variety of sensing modalities have been employed in lung biopsy for bronchoscope navigation. For example, electromagnetic (EM) navigation is based on registration with an anatomical model constructed using pre-operative CT scan; live camera vision provides a direct view for operator to drive a bronchoscope as where the image data is also used in localization by registering the images with the pre-operative CT scan; fluoroscopy from a mobile C-arm fluoroscopy can be used to observe the catheter and the anatomy in real-time; tomosynthesis which is a partial 3D reconstruction based on video of X-ray at varying angles can reveal a lesion, where the lesion can be overlaid on the live fluoroscopic view during navigation or targeting; endobronchial ultrasound (EBUS) has been used to visualize a lesion; robotic kinematics is useful in localizing the tip of the bronchoscope when the catheter is robotically controlled. However, each of the technologies may not be able to provide localization accuracy sufficient enough to navigate the bronchoscope reliably to reach a small lesion in the lung.

Recognized herein is a need for a minimally invasive system that allows for performing surgical procedures or diagnostic operations with improved sensing and localization capability. The present disclosure provides systems and methods allowing for early lung cancer diagnosis and treatment with improved localization accuracy and reliability. In particular, the present disclosure provides a bronchoscopy device with multimodal sensing features by combining multiple sensing modalities using a unique fusion framework. The bronchoscope may combine electromagnetic (EM) sensor, direct imaging device, kinematics data, tomosynthesis and ultrasound imaging using a dynamic fusion framework allowing for small lung modules to be identified specifically outside the airways and automatically steer the bronchoscope towards the target. In some cases, the multiple sensing modalities are dynamically fused based on a real-time confidence score or uncertainty associated with each modality. For example, when a camera view is blocked, or when the quality of the sensor data is not good enough to identify the location of an object, the corresponding modality may be assigned a low confidence score. In some cases, when an electromagnetic (EM) system is used, real-time imaging (e.g., tomosynthesis, EBUS, live camera) may be employed to provide corrections to EM navigation thereby enhancing the localization accuracy.

Additionally, conventional endoscope systems may lack of capability for recovering scope orientation or roll sensing. The present disclosure provides methods and systems with real-time roll detection to recover the orientation of the scope. In particular, a roll detection algorithm is provided to detect the orientation of an imaging device located at the distal end of a flexible catheter. The roll detection algorithm may utilize real-time registration and fluoroscopic image data. This may beneficially avoid the use of a six degrees of freedom sensor (e.g., 6 degree-of-freedom (DOF) EM sensor). In an alternative method, the roll detection may be achieved by using a radiopaque marker on a distal end of a catheter and real-time radiography, such as fluoroscopy.

In an aspect, a method is provided for navigating an endoscopic device through an anatomical luminal network of a patient. The method comprises: (a) commanding a distal tip of an articulating elongate member to move along a pre-determined path; (b) concurrent with (a), collecting positional sensor data and kinematics data; and (c) computing an estimated roll angle of the distal tip based on the positional sensor data and the kinematics data.

In some embodiments, the pre-determined path comprises a straight trajectory. In some embodiments, the pre-determined path comprises a non-straight trajectory.

In some embodiments, the positional sensor data is captured by an electromagnetic (EM) sensor. In some cases, the EM sensor does not measure a roll orientation. In some embodiments, the positional sensor data is obtained from an imaging modality.

In some embodiments, computing the estimated roll angle comprises applying a registration algorithm to the positional sensor data and kinematics data. In some embodiments, the method further comprises evaluating an accuracy of the estimated roll angle.

In another aspect, a method is provided for navigating an endoscopic device through an anatomical luminal network of a patient. The method comprises: (a) attaching a radiopaque maker to a distal end of the endoscopic device; (b) capturing a fluoroscopic image data of the endoscopic device while the endoscopic device is in motion; and (c) reconstructing an orientation of the distal end of the endoscopic device by processing the fluoroscopic image data using a machine learning algorithm trained model.

In some embodiments, the orientation includes a roll angle of the distal end of the endoscopic device. In some embodiments, the machine learning algorithm is deep learning network. In some embodiments, the distal end of the endoscopic device is articulatable and rotatable.

In another aspect, a method is provided for navigating an endoscopic device through an anatomical luminal network of a patient using a multi-modal framework. The method comprises: (a) receiving input data from a plurality of sources including positional sensor data, image data captured by a camera, fluoroscopic image data, ultrasound image data, and kinematics data; (b) determining a confidence score for each of the plurality of sources; (c) generating an input feature data based at least in part on the confidence score and the input data; and (d) processing the input feature data using a machine learning algorithm trained model to generate a navigation output for steering a distal end of the endoscopic device.

In some embodiments, the positional sensor data is captured by an EM sensor attached to the distal end of the endoscopic device. In some embodiments, the camera is embedded to the distal end of the endoscopic device. In some embodiments, the fluoroscopic image data is obtained using tomosynthesis techniques.

In some embodiments, the input data is obtained from the plurality of sources concurrently and is aligned with respect to time. In some embodiments, the ultrasound image data is captured by an array of ultrasound transducers. In some embodiments, the kinematics data is obtained from a robotic control unit of the endoscopic device.

In some embodiments, the navigation output comprises a control command to an actuation unit of the endoscopic device. In some embodiments, the navigation output comprises a navigation guidance to be presented to an operator of the endoscopic device. In some embodiments, the navigation output comprises a desired navigation direction.

In another aspect, a method is provided for compensating a respiratory motion during navigating an endoscopic device through an anatomical luminal network of a patient. The method comprises: (a) capturing positional data during navigating the endoscopic device through the anatomical luminal network; (b) creating a respiratory motion model based on the positional data with aid of a machine learning algorithm trained model, wherein the respiratory motion model is created by distinguishing the respiratory motion from a navigational motion of the endoscopic device; and (c) generating a command to steer a distal portion of the endoscopic device by compensating the respiratory motion using the created respiratory motion model.

In some embodiments, the positional data is captured by an EM sensor located at the distal portion of the endoscopic device. In some embodiments, the machine learning algorithm is a deep learning network. In some embodiments, the positional data is smoothed and decimated.

It should be noted that the provided endoscope systems can be used in various minimally invasive surgical procedures, therapeutic or diagnostic procedures that involve various types of tissue including heart, bladder and lung tissue, and in other anatomical regions of a patient’s body such as a digestive system, including but not limited to the esophagus, liver, stomach, colon, urinary tract, or a respiratory system, including but not limited to the bronchus, the lung, and various others.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates examples of rotation frames.

FIG. 2 shows an example of a calibration procedure.

FIG. 3 shows result of an example of a calibration process.

FIG. 4 shows a scope in a tube lumen in an experiment setup.

FIG. 5 shows an example of a radiopaque marker attached to the catheter tip for pose estimation.

FIG. 6 schematically illustrates an intelligent fusion framework for a multimodal navigation system.

FIG. 7 illustrates an example of calculating compensation for respiratory motion.

FIG. 8 shows an example of a robotic endoscope system supported by a robotic support system.

FIG. 9 shows an example of an instrument driving mechanism providing mechanical interface to the handle portion of the robotic endoscope.

### DETAILED DESCRIPTION OF THE INVENTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

While exemplary embodiments will be primarily directed at a bronchoscope, one of skill in the art will appreciate that this is not intended to be limiting, and the devices described herein may be used for other therapeutic or diagnostic procedures and in other anatomical regions of a patient’s body such as a digestive system, including but not limited to the esophagus, liver, stomach, colon, urinary tract, or a respiratory system, including but not limited to the bronchus, the lung, and various others.

The embodiments disclosed herein can be combined in one or more of many ways to provide improved diagnosis and therapy to a patient. The disclosed embodiments can be combined with existing methods and apparatus to provide improved treatment, such as combination with known methods of pulmonary diagnosis, surgery and surgery of other tissues and organs, for example. It is to be understood that any one or more of the structures and steps as described herein can be combined with any one or more additional structures and steps of the methods and apparatus as described herein, the drawings and supporting text provide descriptions in accordance with embodiments.

Although the treatment planning and definition of diagnosis or surgical procedures as described herein are presented in the context of bronchoscope, pulmonary diagnosis or surgery, the methods and apparatus as described herein can be used to treat any tissue of the body and any organ and vessel of the body such as brain, heart, lungs, intestines, eyes, skin, kidney, liver, pancreas, stomach, uterus, ovaries, testicles, bladder, ear, nose, mouth, soft tissues such as bone marrow, adipose tissue, muscle, glandular and mucosal tissue, spinal and nerve tissue, cartilage, hard biological tissues such as teeth, bone and the like, as well as body lumens and passages such as the sinuses, ureter, colon, esophagus, lung passages, blood vessels and throat.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein a processor encompasses one or more processors, for example a single processor, or a plurality of processors of a distributed processing system for example. A controller or processor as described herein generally comprises a tangible medium to store instructions to implement steps of a process, and the processor may comprise one or more of a central processing unit, programmable array logic, gate array logic, or a field programmable gate array, for example. In some cases, the one or more processors may be a programmable processor (e.g., a central processing unit (CPU) a graphic processing unit (GPU), or a microcontroller), digital signal processors (DSPs), a field programmable gate array (FPGA) and/or one or more Advanced RISC Machine (ARM) processors. In some cases, the one or more processors may be operatively coupled to a non-transitory computer readable medium. The non-transitory computer readable medium can store logic, code, and/or program instructions executable by the one or more processors unit for performing one or more steps. The non-transitory computer readable medium can include one or more memory units (e.g., removable media or external storage such as an SD card or random access memory (RAM)). One or more methods or operations disclosed herein can be implemented in hardware components or combinations of hardware and software such as, for example, ASICs, special purpose computers, or general purpose computers.

As used herein, the terms distal and proximal may generally refer to locations referenced from the apparatus, and can be opposite of anatomical references. For example, a distal location of a bronchoscope or catheter may correspond to a proximal location of an elongate member of the patient, and a proximal location of the bronchoscope or catheter may correspond to a distal location of the elongate member of the patient.

An endoscope system as described herein, includes an elongate portion or elongate member such as a catheter. The terms “elongate member” and “catheter” are used interchangeably throughout the specification unless contexts suggest otherwise. The elongate member can be placed directly into the body lumen or a body cavity. In some embodiments, the system may further include a support apparatus such as a robotic manipulator (e.g., robotic arm) to drive, support, position or control the movements and/or operation of the elongate member. Alternatively or in addition to, the support apparatus may be a hand-held device or other control devices that may or may not include a robotic system. In some embodiments, the system may further include peripheral devices and subsystems such as imaging systems that would assist and/or facilitate the navigation of the elongate member to the target site in the body of a subject.

In some embodiments, the provided systems and methods of the present disclosure may include a multi-modal sensing system which may implement at least a positional sensing system such as electromagnetic (EM) sensor, fiber optic sensors, and/or other sensors to register and display a medical implement together with preoperatively recorded surgical images thereby locating a distal portion of the endoscope with respect to a patient body or global reference frame. The position sensor may be a component of an EM sensor system including one or more conductive coils that may be subjected to an externally generated electromagnetic field. Each coil of EM sensor system used to implement positional sensor system then produces an induced electrical signal having characteristics that depend on the position and orientation of the coil relative to the externally generated electromagnetic field. In some cases, an EM sensor system used to implement the positional sensing system may be configured and positioned to measure at least three degrees of freedom e.g., three position coordinates X, Y, Z. Alternatively or in addition to, the EM sensor system may be configured and positioned to measure five degrees of freedom, e.g., three position coordinates X, Y, Z and two orientation angles indicating pitch and yaw of a base point. In some cases, the roll angle may be provided by including a MEMS-based gyroscopic sensor and/or accelerometer. However, in the case when the gyroscope or the accelerometer is not available, the roll angle may be recovered by a proprietary roll detection algorithm as described later herein.

The present disclosure provides various algorithms and methods for roll detection or estimating catheter pose. The provided methods or algorithms may beneficially allow for catheter pose estimation without using six-DOF sensor. Additionally, the provided methods and algorithms can be easily integrated or applied to any existing systems or devices lack of the roll detection capability without requesting additional hardware or modification to the underlying system.

### Roll Detection Algorithm

The present disclosure provides an algorithm for real-time scope orientation measurement and roll detection. The algorithm provided herein can be used for detecting a roll orientation for any robotically actuated/controlled flexible device. In some embodiments, the algorithm may include a “Wiggle” method for generating an instantaneous roll estimate for the catheter tip. The roll detection algorithm may include a protocol of automated catheter tip motion while the robotic system collects EM sensor data and kinematic data. In some cases, the kinematics data may be obtained from a robotic control unit of the endoscopic device.

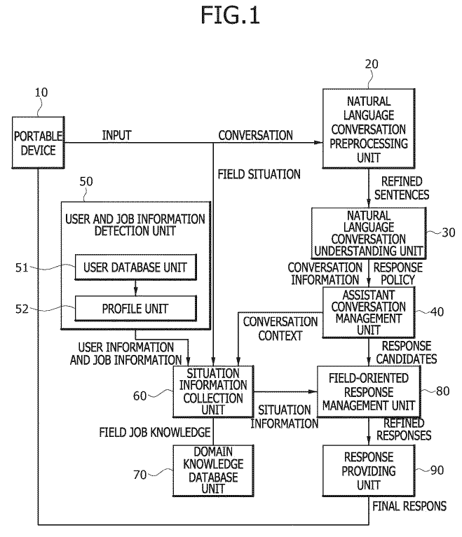

FIG. 1 illustrates examples of rotation frames 100 for a catheter tip 105. In the illustrated example, a camera 101 and one or more illuminating devices (e.g. LED or fiber-based light) 103 may be embedded in the catheter tip. A camera may comprise imaging optics (e.g. lens elements), image sensor (e.g. CMOS or CCD), and illumination (e.g. LED or fiber-based light).

In some embodiments, the catheter 110 may comprise a shaft 111, an articulation (bending) section 107 and a steerable distal portion or catheter tip 105. The articulation section (bending section) 107 connects the steerable distal portion to the shaft 111. For example, the articulation section 107 may be connected to the distal tip portion at a first end, and connected to a shaft portion at a second end or at the base 109. The articulation section may be articulated by one or more pull wires. For example, the distal end of the one or more pull wires may be anchored or integrated to the catheter tip 105, such that operation of the pull wires by the control unit may apply force or tension to the catheter tip 105 thereby steering or articulating (e.g., up, down, pitch, yaw, or any direction in-between) the distal portion (e.g., flexible section) of the catheter.

The rotation frames and rotation matrix that are utilized in the roll detection algorithm are illustrated in the FIG. 1 and are defined as below:

$${R}_{s}^{e m}$$

: real-time EM sensor data provides the relative rotation of the EM sensor frame i.e., ‘s’ with respect to the static EM field generator frame ‘em’;

$${R}_{c t}^{c b}$$

: real-time kinematics data provides the relative rotation of the catheter (e.g., bronchoscope) tip “ct” with respect to the catheter base “cb”. In some cases, the pose of “ct” may be dictated by the pull lengths of a pull-wire.

$${R}_{c b}^{e m}$$

: the result of the registration of the “cb” frame provides the relative rotation of the catheter (e.g., bronchoscope) base frame “cb” with respect to the static EM field generator frame ‘em’.

$${R}_{s}^{c t}$$

: the relative orientation of the EM sensor ‘s’ with respect to the catheter tip frame ‘ct’ can be obtained from a calibration procedure. In some cases,

$${R}_{s}^{c t}$$

may be repeatable across standard or consistent manufacturing the tip assembly.

As described above, the relative orientation of the EM sensor ‘s’ with respect to the catheter tip frame ‘ct’, ie,

$${R}_{s}^{c t}$$

can be obtained from a calibration procedure. In an exemplary process, the standard point-coordinate registration (e.g., least-squares fitting of 3D point sets) may be applied. The exemplary calibration process may include below operations:
(1) fix the base of the articulation section 109 of a catheter to a surface such that the endoscope is in the workspace of the magnetic field generator;
(2) record EM sensor data as well as the kinematic pose of the tip of the endoscope;
(3) articulate the endoscope around its reachable workspace;
(4) post-processing: synchronizing EM and kinematic data;
(5) post-processing: apply a registration algorithm to obtain a rotation matrix
$${R}_{c b}^{e m}$$
(6) calculate rotations to obtain the final relative rotation matrix
$${R}_{c t}^{e m} = {R}_{c b}^{e m} {R}_{c t - s t r a i g h t}^{c b}$$
. The rotation matrix
$${R}_{c t - s t r a i g h t}^{c b}$$
presents the identity matrix when the endoscope is straight. Finally,
$${R}_{s}^{c t} = {( {R}_{c t}^{e m} )}^{T} {R}_{s - s t r a i g h t}^{e m}$$
, where the rotation matrix
$${R}_{s - s t r a i g h t}^{e m}$$
represents the rotation of the EM sensor when the scope is straight.

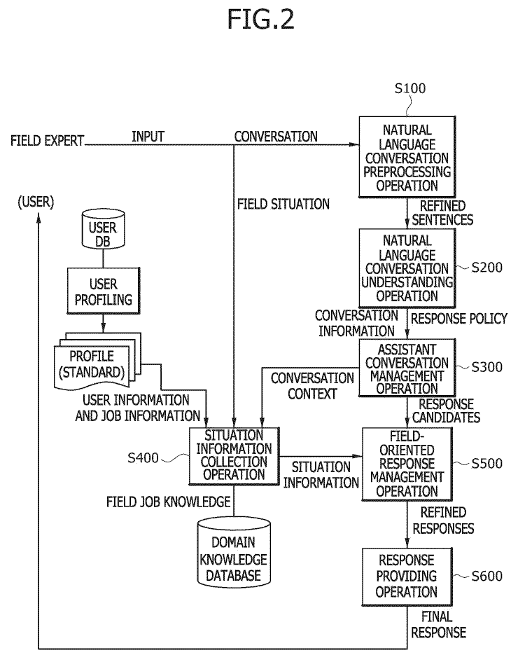

FIG. 2 shows an example of a calibration procedure. The catheter tip is moved (e.g., articulated) while the EM data and kinematic data is collected. In some cases, the calibration procedure may be conducted autonomously without human intervention. For example, articulation of the catheter tip may be automatically performed by executing a pre-determined calibration program. Alternatively or additionally, a user may be permitted to move the catheter tip via a controller. A registration algorithm as described above is applied to compute a relative rotation between the EM sensor located at the tip with respect to the kinematic tip frame.

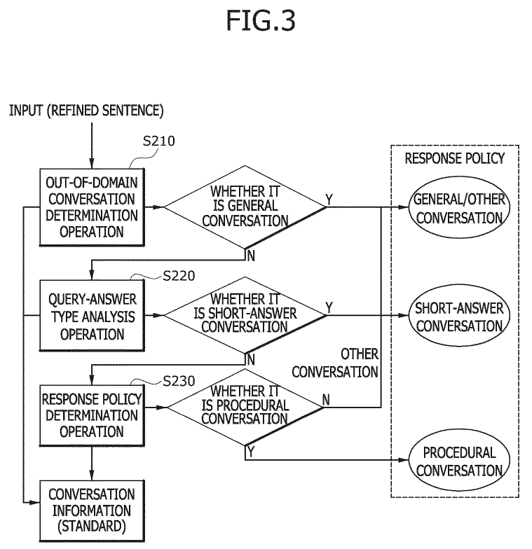

FIG. 3 shows the result of an example of a calibration process. The calibration process may be illustrated in visualization representation to provide real-time visualization of the registration procedure. The calibration process/result can be presented to a user in various forms. As illustrated in the figure, the visualization representation may be a plot showing the calibration process provide an accurate and real-time calibration result. For example, the plot shows that the z-axis of the endoscope base frame is in the approximated direction of the scope-tip heading direction as expected 301. A second observation 303 shows the x-axis of the endoscope base frame is directed away from the EM frame which is an expected result since the scope-tip is oriented such that the camera is closer to the EM field generator. A third observation 305 shows that the x-axis of the “s” frame is properly aligned with the scope-tip heading direction. In some cases, a visual indicator (e.g., textual description or visual indicator) of the calibration observation or result as described above may be displayed to the user on a user interface.

In some embodiments, the roll detection algorithm may comprise an algorithm based on point-coordinates registration. Similar to the aforementioned calibration procedure, this algorithm is dependent on a simple point-coordinate registration. In some cases, instead of wiggling the catheter tip within its workspace (i.e., along non-straight trajectories), calibration can be conducted by commanding the tip to translate along a straight trajectory. The present algorithm may allow for calibration using a straight trajectory (instead of wiggling along a non-straight trajectory) which beneficially shortens the duration of calibration. In an exemplary process, the algorithm may include operations as below:

(1) conduct tip motion by moving the catheter tip around its workspace. EM sensor data and kinematic data is collected while the catheter tip is moved according to a pre-determined path such as wiggling the tip around or following a command to move along a path (e.g., translate along a short straight trajectory).

(2) compute rotational matrix

$${R}_{c b}^{e m}$$

by applying a registration algorithm to the EM sensor data to obtain the rotation matrix

$${R}_{c b}^{e m}$$

. The method can be the same as described above. The output of this registration is the

$${R}_{c b}^{e m}$$

. The position data (e.g., EM sensor) is the input to the algorithm, and the output is an estimated orientation of the endoscope shaft that includes roll orientation. In this way, the relative orientation

$${R}_{c b}^{e m}$$

between the base frames of both sets of position data (e.g., (1) positions of the kinematic tip frame, respective to the kinematic base frame and (2) positions of an EM-sensor that is embedded in the endoscope tip, respective to the EM-field generator coordinate system) can be obtained with a registration process as described above.

(3) reconstruct the expected kinematic catheter tip frame using the EM sensor data. The method may recover an estimated mapping

$${R}_{e m - r e c o n s t r u c t e d}^{c t}$$

. In an ideal scenario, the estimated mapping

$${R}_{e m - r e c o n s t r u c t e d}^{c t}$$

may be identical to the kinematic mapping

$${R}_{c t}^{c b}$$

(that contains no EM information). A difference between the estimated kinematic mapping (based on positional sensor data) and the kinematic mapping (based on kinematics data) may indicate an error in the mapping rotational matrix

$${R}_{c b}^{e m}$$

. By comparing the two kinematics mappings, the presented method is capable of quantitively evaluating the performance (e.g., accuracy) of calculating the endoscope shaft orientation.

The expected kinematic catheter tip frame can be estimated using below equation:

$${R}_{e m - r e c o n s t r u c t e d}^{c t} = {R}_{s}^{c t} {( {R}_{s}^{e m} )}^{T}$$

This rotation matrix represents the orientation of the kinematic tip frame respective to the magnetic field generator, i.e., EM coordinate system. This information is otherwise unknown using kinematics only (e.g., due to the flexible/unknown shape of the elongate member).

By mapping the aforementioned orientation (estimated using the output of the registration), the relative orientation between the endoscope tip frame and the endoscope base frame can be recovered using below equation:

$${R}_{c t - r e c o n s t r u c t e d}^{c b} = {\left({R}_{e m - r e c o n s t r u c t e d}^{c t} {R}_{c b}^{e m}\right)}^{T}$$

The expected or estimated kinematic catheter tip frame is expressed with respect to the kinematic base frame. Such expected kinematic catheter tip frame or the estimated rotation of the catheter tip is obtained only using the position information, i.e. the registration process.

The method may further evaluate the performance of the roll calculation algorithm by computing a rotation offset between the kinematic

$${R}_{c t}^{c b}$$

mapping and the reconstructed tip frame

$${R}_{c t - r e c o n s t r u c t e d}^{c b}$$

. As described above, in an ideal case, these rotation matrices may be identical. To evaluate the correctness of the estimated rotation of the catheter tip obtained in the above step, the rotation offset can be computed using below equation:

$${R}_{e r r o r} = {\left({R}_{c t - r e c o n s t r u c t e d}^{c b}\right)}^{T} {R}_{c t}^{c b}$$

The roll error in the reconstruction of the kinematic frame from the EM sensor data can be computed by decomposing the rotation offset into an axis and an angle representation, wherein the angle holds the meaning of the error in the reconstruction of the kinematic frame from EM sensor data. The error angle can be obtained using below equation:

$$\hat{x} , {θ}_{e r r o r} = G e t A x i s A n g l e \left({R}_{e r r o r}\right)$$

Next, the error angle is projected onto the heading-axis of the endoscope to obtain a pure roll error θᵣ:

$${θ}_{r} = {θ}_{e r r o r} \hat{x} . z$$

In some cases, an alternative method may be used to compute the roll error in the last step. The roll error can be computed using a geometric method by projecting the reconstructed catheter tip coordinate frame onto the plane that is defined by the heading of the endoscope tip, i.e. the heading of the endoscope is orthogonal to the plane. The reconstructed catheter tip x-axis can be computed and the roll error can be defined as the angle between the reconstructed x-axis and the x-axis at the kinematic catheter tip using below equations: [0076]

$${\hat{x}}_{r e c o n s t r u c t e d} = {R}_{c t - r e c o n s t r u c t e d}^{c b} \left[; , 0\right]$$

$${\hat{χ}}_{r e c o n s t r u c t e d} = P {\hat{χ}}_{r e c o n s t r u c t e d}$$

, wherein P is a projection matrix defined as P = I₃ - ẑẑ^{T}that projects a vector onto a plane and ẑ=[0, 0, 1].

$${θ}_{r 2} = \text{acos} \left(\frac{{\hat{x}}_{r e c o n s t r u c t e d} . \hat{x}}{\left‖{\hat{x}}_{r e c o n s t r u c t e d}\right‖}\right)$$

### Experiments

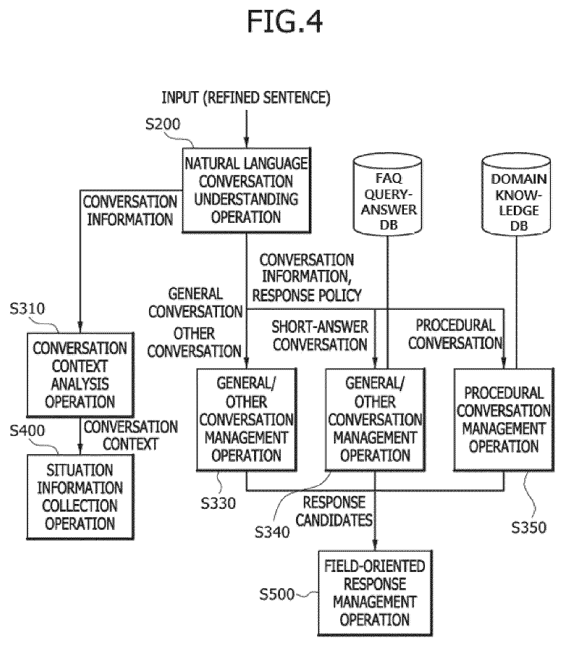

Experiments were conducted by inserting a scope into a tube lumen to simulate the effect of the scope being in a lumen. FIG. 4 shows a scope in a tube lumen in an experiment setup. The proposed algorithm was evaluated on five data sets with a mean computed roll error of 14.8 ± 9.1°. The last two experiments had errors much larger than in the first three experiments. The proposed algorithm was evaluated on five data sets with a mean computed roll error of 14.8 ± 9.1°.

Below is a table of the raw data that is collected or generated from the experiment shown in FIG. 4. θᵣ₂ is the roll angle computed using the alternative method (i.e., geometric method).

| Trial | Data Pts | Motion Time | Max Bend | Orientation Reconstruction | θᵣ (°) | θᵣ₂ (°) |
|---|---|---|---|---|---|---|
| 1 | 380 | 15.2 | 39.9 | 16.4 | 11.3 | 8.8 |
| 2 | 392 | 15.6 | 37.6 | 13.8 | 10.8 | 5.4 |
| 3 | 431 | 17.2 | 45.4 | 11.4 | 0.6 | 8.1 |
| 4 | 401 | 16.0 | 35.7 | 29.9 | 27.5 | 26.8 |
| 5 | 405 | 16.2 | 33.2 | 26.6 | 21.5 | 24.8 |

Other methods may also be employed to for calculating roll orientation. Similarly to the registration process above where two sets of position data are utilized: (1) positions of the kinematic tip frame, respective to the kinematic base frame and (2) positions of an EM-sensor that is embedded in the endoscope tip, respective to the EM-field generator coordinate system. In some embodiments, the EM-sensor may be rigidly fixed in the endoscope tip, as is the kinematic tip frame (albeit not physically), a registration process can be used to compute the relative orientation between the base frames of both sets of position data.

In some cases, instead of using EM sensor data, other sensor data may also be employed to for calculating roll orientation. The non-kinematic position information does not necessarily have to derive from an electromagnetic tracking system. Instead, for example, fluoroscopic image information may be used to capture position information. In conjunction with a registration method described above (e.g., point-coordinate registration or other coordinate registration algorithms), a relative orientation between the endoscope kinematic frame and a reference fluoroscopic coordinate system may be computed. For instance, by mapping motion from the fluoroscopic image data to motion in the kinematics obtained from the driving mechanism motion (e.g., compute the kinematics data and scope tip position based on the fluoroscopic image data), the roll motion can be recovered. In some cases, additional step of mapping image artifacts to coordinate positions may be performed when the imaging modalities (e.g., imaging modalities providing positional data to replace the EM sensor data) do explicitly provide position information in a known coordinate system.

### Catheter Pose Estimation Using Radiopaque Material

In some embodiments, the roll measurement or pose estimation may be achieved using object recognition of radiopaque material. For instance, by disposing a radiopaque pattern at a catheter tip, the orientation of the catheter can be recovered using fluoroscopic imaging and image recognition.

The present methods may be capable of measuring the roll angle along the catheter tip axis when viewed under fluoroscopic imaging. This may beneficially allow for catheter pose estimation without using six-DOF sensor. Additionally, the provided methods may not require user interaction where the catheter orientation can be automatically calculated with aid of fluoroscopic imaging.

Fluoroscopy is an imaging modality that obtains real-time moving images of patient anatomy, medical instruments, and any radiopaque markers within the imaging field using X-rays. Fluoroscopic systems may include C-arm systems which provide positional flexibility and are capable of orbital, horizontal, and/or vertical movement via manual or automated control. Non-C-arm systems are stationary and provide less flexibility in movement. Fluoroscopy systems generally use either an image intensifier or a flat-panel detector to generate two dimensional real-time images of a patient anatomy. Bi-planar fluoroscopy systems simultaneously capture two fluoroscopic images, each from different (often orthogonal) viewpoints. In the presented methods, a radiopaque marker disposed at the tip of the catheter may be visible by the fluoroscopic imaging and is analyzed for estimating a pose of the catheter or the camera.

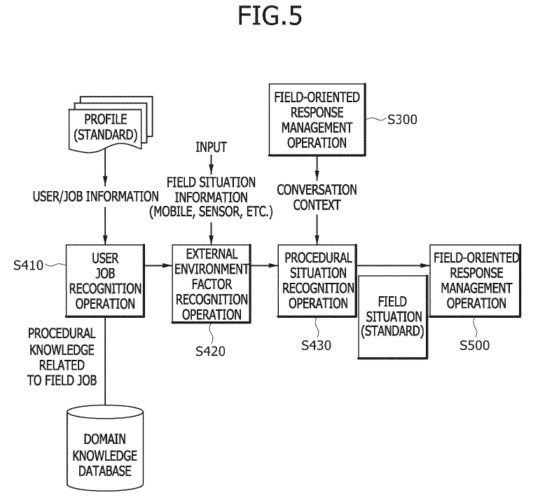

FIG. 5 shows an example of a radiopaque marker 503 attached to the catheter tip 501 for pose estimation. As shown in the figure, a radiopaque pattern is placed on the tip of an endoscope and imaged by fluoroscopic imaging. In some cases, the radiopaque marker may be integrally coupled to an outside surface of the tip of the elongate member. Alternatively, the radiopaque marker may be removably coupled to the elongate member. The fluoroscopic image data may be captured while the endoscopic device is in motion. The radiopaque pattern is visible in the fluoroscopic image data. The fluoroscopic image data may be processed for recovering the orientation of the catheter tip such as using computer vision, machine learning, or other object recognition methods to recognize and analyze the shape of the marker in the fluoroscopic image.

The radiopaque marker may have any pattern, shape or geometrics that is useful for recovering the 3D orientation of the catheter tip. For instance, the pattern may be non-symmetrical with at least three points. In the illustrated example, the radiopaque marker has an “L” shape which is not intended to be limiting. Markers of many shapes and sizes can be employed. In some cases, the markers may have a non-symmetrical shape or pattern with at least three distinguishable points.

Computer vision (CV) techniques or computer vision systems have been used to process 2D image data for constructing 3D orientation or pose of an object. Any other suitable optical methods or image processing techniques may be utilized to recognize and isolate the pattern, as well as associate it with one of the rotational angles. For example, the orientation of the camera or the catheter tip portion can be obtained using methods including, for example, object recognition, stereoscopy, monocular shape-from-motion, shape-from-shading, and Simultaneous Localization and Mapping (SLAM) or other computer vision techniques such as optical flow, computational stereo approaches, iterative method combined with predictive models, machine learning approaches, predictive filtering or any non-rigid registration methods.

In some cases, the optical techniques for predicting the catheter pose or roll angle may employ one or more trained predictive models. In some cases, the input data to be processed by the predictive models may include image or optical data. The image data or video data may be captured by a fluoroscopic system (e.g., C-arm system) and the roll orientation may be recovered in real-time while the image or optical data is collected.

The one or more predictive models can be trained using any suitable deep learning networks. For example, the deep learning network may employ U-Net architecture which is essentially a multi-scale encoder-decoder architecture, with skip-connections that forward the output of each of the encoder layers directly to the input of the corresponding decoder layers. As an example of a U-Net architecture, unsampling in the decoder is performed with a pixelshuffle layer which helps reducing gridding artifacts. The merging of the features of the encoder with those of the decoder is performed with pixel-wise addition operation resulting in a reduction of memory requirements. The residual connection between the central input frame and the output is introduced to accelerate the training process.

The deep learning model can employ any type of neural network model, such as a feedforward neural network, radial basis function network, recurrent neural network, convolutional neural network, deep residual learning network and the like. In some embodiments, the deep learning algorithm may be convolutional neural network (CNN). The model network may be a deep learning network such as CNN that may comprise multiple layers. For example, the CNN model may comprise at least an input layer, a number of hidden layers and an output layer. A CNN model may comprise any total number of layers, and any number of hidden layers. The simplest architecture of a neural network starts with an input layer followed by a sequence of intermediate or hidden layers, and ends with output layer. The hidden or intermediate layers may act as learnable feature extractors, while the output layer may output the improved image frame. Each layer of the neural network may comprise a number of neurons (or nodes). A neuron receives input that comes either directly from the input data (e.g., low quality image data etc.) or the output of other neurons, and performs a specific operation, e.g., summation. In some cases, a connection from an input to a neuron is associated with a weight (or weighting factor). In some cases, the neuron may sum up the products of all pairs of inputs and their associated weights. In some cases, the weighted sum is offset with a bias. In some cases, the output of a neuron may be gated using a threshold or activation function. The activation function may be linear or non-linear. The activation function may be, for example, a rectified linear unit (ReLU) activation function or other functions such as saturating hyperbolic tangent, identity, binary step, logistic, arcTan, softsign, parameteric rectified linear unit, exponential linear unit, softPlus, bent identity, softExponential, Sinusoid, Sinc, Gaussian, sigmoid functions, or any combination thereof. During a training process, the weights or parameters of the CNN are tuned to approximate the ground truth data thereby learning a mapping from the input raw image data to the desired output data (e.g., orientation of an object in a 3D scene).

### Hybrid Imaging and Navigation

The endoscope system of the present disclosure may combine multiple sensing modalities to provide enhanced navigation capability. In some embodiments, the multimodal sensing system may comprise at least positional sensing (e.g., EM sensor system), direct vision (e.g., camera), ultrasound imaging, and tomosynthesis.

As described above, electromagnetic (EM) navigation is based on registration with an anatomical model constructed using pre-operative CT scan; live camera vision provides a direct view for operator to drive a bronchoscope as where the image data is also used in localization by registering the images with the pre-operative CT scan; fluoroscopy from a mobile C-arm fluoroscopy can be used to observe the catheter and the anatomy in real-time; tomosynthesis which is a partial 3D reconstruction based on video of X-ray at varying angles can reveal a lesion, where the lesion can be overlaid on the live fluoroscopic view during navigation or targeting; endobronchial ultrasound (EBUS) has been used to visualize a lesion; robotic kinematics is useful in localizing the tip of the bronchoscope when the catheter is robotically controlled. In some cases, the kinematics data may be obtained from a robotic control unit of the endoscopic device

In some cases, the endoscope system may implement a positional sensing system such as electromagnetic (EM) sensor, fiber optic sensors, and/or other sensors to register and display a medical implement together with preoperatively recorded surgical images thereby locating a distal portion of the endoscope with respect to a patient body or global reference frame. The position sensor may be a component of an EM sensor system including one or more conductive coils that may be subjected to an externally generated electromagnetic field. Each coil of EM sensor system used to implement positional sensor system then produces an induced electrical signal having characteristics that depend on the position and orientation of the coil relative to the externally generated electromagnetic field. In some cases, an EM sensor system used to implement the positional sensing system may be configured and positioned to measure at least three degrees of freedom e.g., three position coordinates X, Y, Z. Alternatively or in addition to, the EM sensor system may be configured and positioned to measure six degrees of freedom, e.g., three position coordinates X, Y, Z and three orientation angles indicating pitch, yaw, and roll of a base point or five degrees of freedom, e.g., three position coordinates X, Y, Z and two orientation angles indicating pitch and yaw of a base point.

The direct vision may be provided by an imaging device such as a camera. The imaging device may be located at the distal tip of the catheter or elongate member of the endoscope. In some cases, the direction vision system may comprise an imaging device and an illumination device. In some embodiments, the imaging device may be a video camera. The imaging device may comprise optical elements and image sensor for capturing image data. The image sensors may be configured to generate image data in response to wavelengths of light. A variety of image sensors may be employed for capturing image data such as complementary metal oxide semiconductor (CMOS) or charge-coupled device (CCD). The imaging device may be a low-cost camera. In some cases, the image sensor may be provided on a circuit board. The circuit board may be an imaging printed circuit board (PCB). The PCB may comprise a plurality of electronic elements for processing the image signal. For instance, the circuit for a CCD sensor may comprise A/D converters and amplifiers to amplify and convert the analog signal provided by the CCD sensor. Optionally, the image sensor may be integrated with amplifiers and converters to convert analog signal to digital signal such that a circuit board may not be required. In some cases, the output of the image sensor or the circuit board may be image data (digital signals) can be further processed by a camera circuit or processors of the camera. In some cases, the image sensor may comprise an array of optical sensors. As described later herein, the imaging device may be located at the distal tip of the catheter or an independent hybrid probe which is assembled to the endoscope.

The illumination device may comprise one or more light sources positioned at the distal tip of the endoscope or catheter. The light source may be a light-emitting diode (LED), an organic LED (OLED), a quantum dot, or any other suitable light source. In some cases, the light source may be miniaturized LED for a compact design or Dual Tone Flash LED Lighting.

The provided endoscope system may use ultrasound to help guide physicians to a location outside of an airway. For example, a user may use the ultrasound to locate, in real-time a lesion location to guide the endoscope to a location where a computed tomography (CT) scan revealed the approximate location of a solitary pulmonary nodule. The ultrasound may be a linear endobronchial ultrasound (EBUS), also known as convex probe EBUS, may image to the side of the endoscope device or a radial probe EBUS which images radially 360°. For example, a linear endobronchial ultrasound (EBUS) transducer or transducer array may be located at the distal portion of the endoscope.

The multimodal sensing feature of the present disclosure may include combining the multiple sensing modalities using a unique fusion framework. The bronchoscope may combine electromagnetic (EM) sensor, direct imaging device, tomosynthesis, kinematics data and ultrasound imaging using a dynamic fusion framework allowing for small lung modules to be identified specifically outside the airways and automatically steer the bronchoscope towards the target. In some cases, the multiple sensing modalities are dynamically fused based on a real-time confidence score or uncertainty associated with each modality. In some cases, when an electromagnetic (EM) system is used, real-time imaging (e.g., tomosynthesis, EBUS, live camera) may be employed to provide corrections to EM navigation thereby enhancing the localization accuracy.

The provided systems and methods may comprise a multimodal navigation system utilizing machine learning and AI technologies to optimize fusion of multimodal data. In some embodiments, the multimodal navigation system may combine the four or more different sensory modalities i.e., positional sensing (e.g., EM sensor system), direct vision (e.g., camera), ultrasound imaging, kinematics data and tomosynthesis via an intelligent fusion framework.

The intelligent fusion framework may include one or more predictive models can be trained using any suitable deep learning networks as described above. The deep learning model may be trained using supervised learning or semi-supervised learning. For example, in order to train the deep learning network, pairs of datasets with input image data (i.e., images captured by the camera) and desired output data (e.g., navigation direction, pose or location of the catheter tip) may be generated by a training module of the system as training dataset.

Alternatively or in addition to, hand-crafted rules may be utilized by the fusion framework. For example, a confidence score may be generated for each of the different modalities and the multiple data may be combined based on the real-time condition.

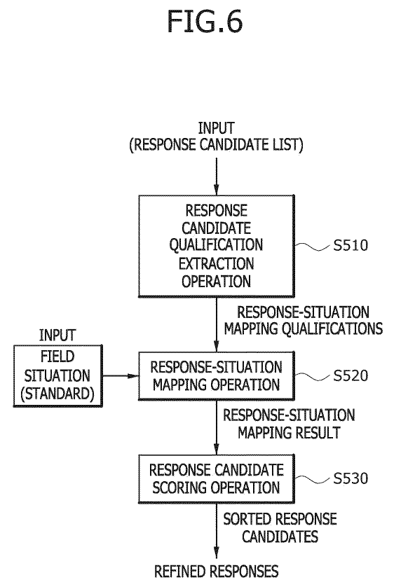

FIG. 6 schematically illustrates an intelligent fusion framework 600 for dynamically controlling the multimodal navigation system, fusing and processing real-time sensory data and robotic kinematics data to generate an output for navigation and various other purposes. In some embodiments, the intelligent fusion framework 600 may comprise a positional sensor 610, an optical imaging device (e.g., camera) 620, a tomosynthesis system 630, an EBUS imaging system 640, a robotic control system 650 to provide robotic kinematics data, a sensor fusion component 660 and an intelligent navigation direction inference engine 670. The positional sensor 610, optical imaging device (e.g., camera) 620, tomosynthesis system 630, EBUS imaging system 640 and the robotic kinematics data 650 can be the same as those described above.

In some embodiments, the output 613 of the navigation engine 670 may include a desired navigation direction or a steering control output signal for steering a robotic endoscope in real-time. In some cases, when the robotic endoscope system is in an autonomous mode, the multimodal navigation system may utilize an artificial intelligence algorithm (e.g., a deep machine learning algorithm) to process the multimodal input data and provide a predicted steering direction and/or steering control signal as output for steering the distal tip of the robotic endoscope. In some instances, e.g., in fully-automated mode, the multimodal navigation system may be configured to guide the advancing endoscope with little or no input from a surgeon or other operator. The output 613 may comprise a desired direction that is translated by a controller of the robotic endoscope system into control signals to control one or more actuation units. Alternatively, the output may include the control commands for the one or more actuation units directly. In some cases, the e.g., in semi-automated mode, the multimodal navigation system may be configured to provide assistance to a surgeon who is actively guiding the advancing endoscope. In such case, the output 613 may include guidance to an operator of the robotic endoscope system.

The output 613 may be generated by the navigation engine 670. In some embodiments, the navigation engine 670 may include an input feature generation module 671 and a trained predictive model 673. A predictive model may be a trained model or trained using machine learning algorithm. The machine learning algorithm can be any type of machine learning network such as: a support vector machine (SVM), a naive Bayes classification, a linear regression model, a quantile regression model, a logistic regression model, a random forest, a neural network, convolutional neural network CNN, recurrent neural network RNN, a gradient-boosted classifier or repressor, or another supervised or unsupervised machine learning algorithm (e.g., generative adversarial network (GAN), Cycle-GAN, etc. ).

The input feature generation module 671 may generate input feature data to be processed by the trained predictive model 673. In some embodiments, the input feature generation module 671 may receive data from the positional sensor 610, optical imaging device (e.g., camera) 620, a tomosynthesis system 630, an EBUS imaging system 640 and robotic kinematics data 650, extract features and generate the input feature data. In some embodiments, the data received from the positional sensor 610, optical imaging device (e.g., camera) 620, tomosynthesis system 630, EBUS imaging system 640 may include raw sensor data (e.g., image data, EM data, tomosynthesis data, ultrasound image, etc.). In some cases, the input feature generation module 671 may pre-process the raw input data (e.g., data alignment) generated by the multiple different sensory systems (e.g., sensors may capture data at different frequency) or from different sources (e.g., third-party application data). For example, data captured by camera, positional sensor (e.g., EM sensor), ultrasound image data, tomosynthesis data may be aligned with respect to time and/or identified features (e.g., lesion). In some cases, the multiple sources of data may be captured concurrently.

The data received from the variety of data sources 610, 620, 630, 640, 650 may include processed data. For example, data from the tomosynthesis system may include reconstructed data or information about a lesion identified from the raw data.

In some cases, the data 611 received from the multimodal data sources may be adaptive to real-time conditions. The sensor fusion component 660 may be operably coupled to the data sources to receive the respective output data. In some cases, the output data produced by the data sources 610, 620, 630, 640, 650 may be dynamically adjusted based on real-time conditions. For instance, the multiple sensing modalities are dynamically fused based on a real-time confidence score or uncertainty associated with each modality. The sensor fusion component 660 may assess the confidence score for each data source and determine the input data to be used for inferring navigation direction. For example, when a camera view is blocked, or when the quality of the sensor data is not good enough to identify the location of an object, the corresponding modality may be assigned a low confidence score. In some cases, the sensor fusion component 660 may weight the data from the multiple sources based on the confidence score. The multiple data may be combined based on the real-time condition. In some cases, when an electromagnetic (EM) system is used, real-time imaging (e.g., tomosynthesis, EBUS, live camera) may be employed to provide corrections to EM navigation thereby enhancing the localization accuracy.

### Respiration Compensation for Electromagnetic (EM)-Based Navigation

While traversing the lung structure, a bronchoscope can be moved by certain offset (e.g., up to two centimeters) due to respiratory motion. A need exists to compensate for the respiratory motion there by allowing a smooth navigation and improved alignment with a target site (e.g., lesion).

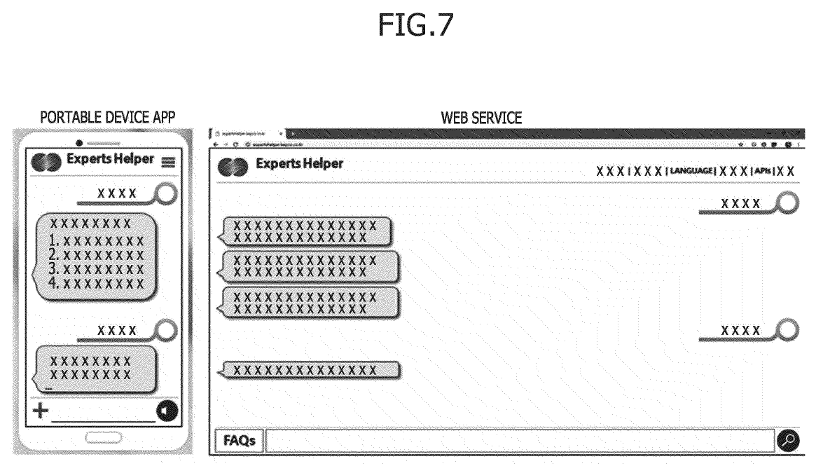

The present disclosure may improve the navigation and location tracking by creating a real-time adaptive model predicting the respiratory motion. In some embodiments, the respiratory motion model may be generated based on positional sensor (e.g., EM sensor) data. FIG. 7 illustrates an example of calculating compensation for respiratory motion.

The sensor data for building the model may be captured while the device with the EM sensor is placed inside a patient body without user operation so the detected motion is substantially the respiratory motion of the patient. Alternatively or in addition to, the sensor data for building the model may be collected while the device is driven or operated such that the collected sensor data may indicate a motion as result of both respiratory motion and the device’s active motion. In some cases, the motion model may be relatively a low order parametric model which can be created by using self-correlation of the sensor signal to identify the cyclic motion frequency and/or using a filter to extract the low frequency motion. Alternatively or in addition to, the model may be created using reference signal. For example, positional sensor located on the patient body, elastic band, ventilator, or audio signal from the ventilator operation may be utilized to provide a reference signal to distinguish the respiratory motion from the raw sensory data.

The method may include preprocessing the positional sensor data by smoothing, decimating, and splitting the positional sensor data into dimensional components. The type, form or format of the time-series positional data may depend on the types of sensors. For example, when the time-series data is collected from a six-DOF EM sensor, the time-series data may be decomposed into X, Y, Z axis. In some cases, the time-series data may be pre-processed and arranged into a three-dimensional numerical array.

The respiratory motion model may be constructed by fitting a defined function dimensionally to the pre-processed sensor data. The constructed model can be used to calculate an offset that is applied to the incoming sensor data to compensate the respiratory motion in real-time. In some cases, the respiratory motion model may be calculated and updated as new sensory data are collected and processed and the updated respiratory motion model may be deployed for use.

In some cases, static information from the lung segmentation may be utilized to distinguish user action from respiratory motion thereby increasing the prediction accuracy. In some cases, the model may be created using machine learning techniques. In some cases, the respiratory motion model is created by distinguishing the respiratory motion from a navigational motion of the endoscopic device with aid of machine learning techniques. Various deep learning models and framework as described elsewhere herein may be used to train the respiratory model. In some cases, the EM sensor data may be pre-processed (e.g., smoothed and decimated) and the pre-processed EM sensor data may be used to generate input features to be processed by the trained model.

The respiratory motion model may be used for planning tool trajectories and/or navigating the endoscope. For example, command for deflecting the distal tip of the scope to follow a pathway of a structure under examination may be generated by compensating the respiratory motion thereby minimizing friction force upon the surrounding tissue. In another example, it is beneficial to time surgical tasks or subtasks (e.g., inserting needle) for the pause between exhaling and inhaling.

In some embodiments, the endoscopic device may be a single-use robotic endoscope. In some cases, only the catheter may be disposable. In some cases, at least a portion of the catheter may be disposable. In some cases, the entire robotic endoscope may be released from an instrument driving mechanism and can be disposed of.

The robotic endoscope described herein may include suitable means for deflecting the distal tip of the scope to follow the pathway of the structure under examination, with minimum deflection or friction force upon the surrounding tissue. For example, control cables or pulling cables are carried within the endoscope body in order to connect an articulation section adjacent to the distal end to a set of control mechanisms at the proximal end of the endoscope (e.g., handle) or a robotic support system. The orientation (e.g., roll angle) of the distal tip may be recovered by the method described above. The navigation control signals may be generated by the navigation system as described above and the control of the motion of the robotic endoscope may have the respiratory compensation capability as described above.

The robotic endoscope system can be releasably coupled to an instrument driving mechanism. The instrument driving mechanism may be mounted to the arm of the robotic support system or to any actuated support system. The instrument driving mechanism may provide mechanical and electrical interface to the robotic endoscope system. The mechanical interface may allow the robotic endoscope system to be releasably coupled to the instrument driving mechanism. For instance, the handle portion of the robotic endoscope can be attached to the instrument driving mechanism via quick install/release means, such as magnets and spring-loaded levels. In some cases, the robotic endoscope may be coupled to or released from the instrument driving mechanism manually without using a tool.

FIG. 8 shows an example of a robotic endoscope system supported by a robotic support system. In some cases, the handle portion may be in electrical communication with the instrument driving mechanism (e.g., instrument driving mechanism 820) via an electrical interface (e.g., printed circuit board) so that image/video data and/or sensor data can be received by the communication module of the instrument driving mechanism and may be transmitted to other external devices/systems. In some cases, the electrical interface may establish electrical communication without cables or wires. For example, the interface may comprise pins soldered onto an electronics board such as a printed circuit board (PCB). For instance, receptacle connector (e.g., the female connector) is provided on the instrument driving mechanism as the mating interface. This may beneficially allow the endoscope to be quickly plugged into the instrument driving mechanism or robotic support without utilizing extra cables. Such type of electrical interface may also serve as a mechanical interface such that when the handle portion is plugged into the instrument driving mechanism, both mechanical and electrical coupling is established. Alternatively or in addition to, the instrument driving mechanism may provide a mechanical interface only. The handle portion may be in electrical communication with a modular wireless communication device or any other user device (e.g., portable/hand-held device or controller) for transmitting sensor data and/or receiving control signals.

As shown in FIG. 8, a robotic endoscope 820 may comprise a handle portion 813 and a flexible elongate member 811. In some embodiments, the flexible elongate member 811 may comprise a shaft, steerable tip and a steerable section as described elsewhere herein. The robotic endoscope may be a single-use robotic endoscope. In some cases, only the catheter may be disposable. In some cases, at least a portion of the catheter may be disposable. In some cases, the entire robotic endoscope may be released from the instrument driving mechanism and can be disposed of. The endoscope may contain varying levels of stiffness along its shaft, as to improve functional operation.

The robotic endoscope can be releasably coupled to an instrument driving mechanism 820. The instrument driving mechanism 820 may be mounted to the arm of the robotic support system or to any actuated support system as described elsewhere herein. The instrument driving mechanism may provide mechanical and electrical interface to the robotic endoscope 820. The mechanical interface may allow the robotic endoscope 820 to be releasably coupled to the instrument driving mechanism. For instance, the handle portion of the robotic bronchoscope can be attached to the instrument driving mechanism via quick install/release means, such as magnets and spring-loaded levels. In some cases, the robotic bronchoscope may be coupled or released from the instrument driving mechanism manually without using a tool.

FIG. 9 shows an example of an instrument driving mechanism 920 providing mechanical interface to the handle portion 913 of the robotic endoscope. As shown in the example, the instrument driving mechanism 920 may comprise a set of motors that are actuated to rotationally drive a set of pull wires of the catheter. The handle portion 913 of the catheter assembly may be mounted onto the instrument drive mechanism so that its pulley assemblies are driven by the set of motors. The number of pulleys may vary based on the pull wire configurations. In some cases, one, two, three, four, or more pull wires may be utilized for articulating the catheter.

The handle portion may be designed allowing the robotic endoscope to be disposable at reduced cost. For instance, classic manual and robotic endoscope may have a cable in the proximal end of the endoscope handle. The cable often includes illumination fibers, camera video cable, and other sensors fibers or cables such as electromagnetic (EM) sensors, or shape sensing fibers. Such complex cable can be expensive adding to the cost of the bronchoscope. The provided robotic endoscope may have an optimized design such that simplified structures and components can be employed while preserving the mechanical and electrical functionalities. In some cases, the handle portion of the robotic endoscope may employ a cable-free design while providing a mechanical/electrical interface to the catheter.

In some case, the handle portion may be housing or comprise components configured to process image data, provide power, or establish communication with other external devices. In some cases, the communication may be wireless communication. For example, the wireless communications may include Wi-Fi, radio communications, Bluetooth, IR communications, or other types of direct communications. Such wireless communication capability may allow the robotic bronchoscope function in a plug-and-play fashion and can be conveniently disposed after single use. In some cases, the handle portion may comprise circuitry elements such as power sources for powering the electronics (e.g. camera and LED light source) disposed within the robotic bronchoscope or catheter.

The handle portion may be designed in conjunction with the catheter such that cables or fibers can be eliminated. For instance, the catheter portion may employ a design having working channel allowing instruments to pass through the robotic bronchoscope, a vision channel allowing a hybrid probe to pass through, as well as low cost electronics such as a chip-on-tip camera, illumination sources such as light emitting diode (LED) and EM sensors located at optimal locations in accordance with the mechanical structure of the catheter. This may allow for a simplified design of the handle portion. For instance, by using LEDs for illumination, the termination at the handle portion can be based on electrical soldering or wire crimping alone. For example, the handle portion may include a proximal board where the camera cable, LED cable, and EM sensor cable terminate while the proximal board connects to the interface of the handle portion and establishes the electrical connections to the instrument driving mechanism. As described above, the instrument driving mechanism is attached to the robot arm (robotic support system) and provides a mechanical and electrical interface to the handle portion. This may advantageously improve the assembly and implementation efficiency as well as simplify the manufacturing process and cost. In some cases, the handle portion along with the catheter may be disposed of after a single use.

The robotic endoscope may have compact configuration of the electronic elements disposed at the distal portion. Design for the distal tip/portion design and the navigation systems/methods can include those described in the PCT/US2020/65999, entitled “systems and methods for robotic bronchoscopy”, which is incorporated by reference herein in its entirety.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for navigating an endoscopic device through an anatomical luminal network of a patient, the method comprising:
(a) commanding a distal tip of an articulating elongate member to move along a pre-determined path;
(b) concurrent with (a), collecting positional sensor data and kinematics data; and
(c) computing an estimated roll angle of the distal tip based on the positional sensor data and the kinematics data.

2. The method of claim 1, wherein the pre-determined path comprises only a straight trajectory.

3. The method of claim 1, wherein the pre-determined path comprises a non-straight trajectory.

4. The method of claim 1, wherein the positional sensor data is captured by an electromagnetic (EM) sensor that is located at the distal tip of the articulating elongate member.

5. The method of claim 4, wherein the EM sensor does not measure a roll orientation.

6. The method of claim 1, wherein the positional sensor data is obtained from an imaging modality.

7. The method of claim 1, wherein computing the estimated roll angle comprises: i) synchronizing the positional sensor data and the kinematics data, and ii) applying a registration algorithm to obtain a rotation matrix.

8. The method of claim 1, further comprising evaluating an accuracy of the estimated roll angle.

9. The method of claim 8, wherein the accuracy is calculated based at least in part on a first kinematics mapping based on the positional sensor data and a second kinematics mapping based on the kinematics data.

10. A method for navigating an endoscopic device through an anatomical luminal network of a patient, the method comprising:
(a) receiving input data from a plurality of sources including positional sensor data, image data captured by a camera, fluoroscopic image data, ultrasound image data, and kinematics data;
(b) determining a confidence score for each of the plurality of sources;
(c) generating an input feature data based at least in part on the confidence score and the input data; and
(d) processing the input feature data using a machine learning algorithm trained model to generate a navigation output for steering a distal end of the endoscopic device.

11. The method of claim 10, wherein the positional sensor data is captured by an EM sensor attached to the distal end of the endoscopic device.

12. The method of claim 10, wherein the camera is embedded to the distal end of the endoscopic device.

13. The method of claim 10, wherein the fluoroscopic image data is obtained using tomosynthesis techniques.

14. The method of claim 10, wherein the input data is obtained from the plurality of sources concurrently and is aligned with respect to time.

15. The method of claim 10, wherein the ultrasound image data is captured by an array of ultrasound transducers.

16. The method of claim 10, wherein the kinematics data is obtained from a robotic control unit of the endoscopic device.

17. The method of claim 10, wherein the navigation output comprises a control command to an actuation unit of the endoscopic device.

18. The method of claim 10, wherein the navigation output comprises a navigation guidance to be presented to an operator of the endoscopic device or a desired navigation direction.

19. The method of claim 10, further comprising creating a respiratory motion model based on the positional data with aid of a machine learning algorithm trained model, wherein the respiratory motion model is created by distinguishing the respiratory motion from a navigational motion of the endoscopic device.

20. The method of claim 19, further comprising updating the navigation output by compensating the respiratory motion using the created respiratory motion model.
